(19) United States
(12) Patent Application Publication
(10) Pub. No.: US 2007/0123589 A1
(43) Pub. Date: May 31, 2007

(54) Method for producing chiral or enantiomer-enriched beta-amino acids, aldehydes, ketones and gama-amino alcohols

(76) Inventors: Walther Jary, Steinbach am Attersee (AT); Ben De Lange, Munstergeleen (NL); Quirinus Bernardus Broxterman, Munstergeleen (NL); Peter Pochlauer, Linz (AT); Marcellas Van Der Sluis, Groningen (NL); Patrick Uiterweerd, Groningen (NL); Heinz Falk, Linz (AT); Gerhard Zuckerstatter, Puch bei Salsburg (AT)

Correspondence Address: NIXON & VANDERHYE, PC, 901 NORTH GLEBE ROAD, 11TH FLOOR, ARLINGTON, VA, 22203, US

(21) Appl. No.: 10/582,671

(22) PCT Filed: Nov. 25, 2004
(86) PCT No.: PCT/EP04/13354

§ 371 Date: Jun. 12, 2006

(30) Foreign Application Priority Data
Dec. 19, 2003 (AT) A 2051/2003
May 28, 2004 (AT) A 929/2004

(51) Int. Cl. A61K 31/195; A61K 31/13; C07C 227/02

(52) U.S. Cl. 514/561; 562/553; 564/502; 564/503; 514/666; 514/667

(57) ABSTRACT

The invention relates to an improved method for producing chiral or enantiomer-enriched beta amino acids, -aldehydes, -ketones and gamma-amino alcohols, during which an allyl amine of formula (I), in which: R1 represents an alkyl radical, a cycloalkyl radical, an alkyl radical, a heterocycle radical or a condensed or bridged ring system; R2, R3, R4 and R5, independent of one another, can represent H or an alkyl radical, a cyclo alkyl radical, an aryl radical, a heterocycle radical or a condensed or bridged ring system or radicals R1, R2, R3 and R4, together, form ring systems that can optionally contain one or more heteroatoms, whereby radicals R1, R2, R3, R4 and R5 can be substituted once or a number of times, and; R6 represents H or an N-protective group, is transformed; a) by ozonolysis in a solvent and; b) subsequent decomposition of the peroxide-containing solution by means of an oxidizing agent or reductive reprocessing, into the corresponding amino compound of formula (II), in which R1, R2, R3, R4 and R6 are defined as above, and; A, according to the reprocessing, represents a radical of formula COOH, —C(OH)R5 or —C(O)R5, whereby R5 is defined as above.

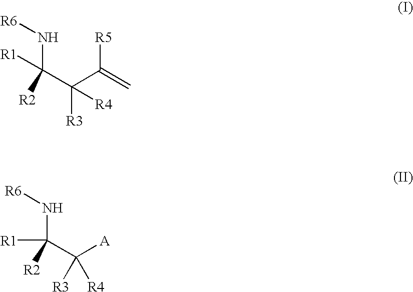

The invention relates to an improved method for preparing chiral or enantiomer-enriched beta-amino acids, aldehydes, ketones and gamma-amino alcohols starting from optionally N-protected homoallylamines, chiral or enantiomer-enriched beta-amino acids, aldehydes, ketones and gamma-amino alcohols are used for example as chiral aid, chiral ligands, precursor for beta-lactams, beta-peptides or as starting material for preparing a wide variety of naturally occurring bioactive substances, as chiral synthons, as inter-mediates in the preparation of pharmaceuticals.

Methods for preparing chiral or enantiomer-enriched beta-amino acids, aldehydes, ketones and gamma-amino alcohols have been disclosed in great number in the literature. Reaction of olefins in methanolic sodium hydroxide solution leads to the corresponding carboxylic esters. These methods can also be used to prepare beta-amino acid esters and are described in the literature: J. A. Marshall, A. W. Garofalo, R. C. Sedrani, Synlettt, 1992, 643-645. b) Oxidative cleavage of mono-, di-, and trisubstituted olefins to methyl esters through ozonolysis in methanolic NaOH, J. A. Marshall, A. W. Garafalo, J. Org. Chem. 1993, 58, 3675-3680. However, the disadvantage of these methods is that ozone is to a very large extent decomposed in the alkaline medium and only a small part of the ozone is available for cleaving the double bond. The longer reaction time resulting therefrom makes these methods extremely uneconomical.

A further disadvantage of this method is that the amino acid esters are obtained. In order to obtain the free amino acids, the ester functionality must be hydrolyzed in a further method step.

According to WO 01/42173, firstly a phenylglycinamide is reacted inter alia with an aldehyde, such as, for instance, isobutyraldehyde and then converted into the corresponding Schiff's base. This Schiff's base is then reacted by reaction with an allylic organometallic compound to give the corresponding allyl compound which is converted by oxidative methods such as, for instance, by ozonolysis, subsequent oxidative treatment and final hydrogenolysis into the desired beta-amino acid. The beta-amino acids are in this case obtained in an amount of about 30% after purification by column chromatography. In order to obtain the corresponding beta-amino alcohol, once again according to WO 01/42173 first the allyl compound is ozonolyzed, followed by reductive work-up, for example using NaBH₄, and protective group elimination by hydrogenolysis. In this case, the corresponding amino alcohol is obtained in a yield of about 47% after purification by column chromatography.

It was an object of the present invention to find an alternative method starting from more easily obtainable allyl precursors and leading to the desired beta-amino acids, aldehydes, ketones or gamma-amino alcohols in yields of 58% to 99%.

The invention accordingly relates to an improved method for preparing chiral or enantiomer-enriched beta-amino acids, aldehydes, ketones or gamma-amino alcohols, which is characterized in that an allylamine of the formula 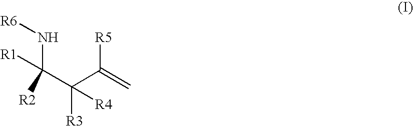
in which R1 is an alkyl radical, a cycloalkyl radical, an aryl radical, a heterocyclic radical or a fused or bridged ring system,
R2, R3, R4 and R5 may independently of one another be H or an alkyl radical, a cycloalkyl radical, an aryl radical, a heterocyclic radical or a fused or bridged ring system,
or the radicals R1, R2, R3 and R4 may form ring systems among themselves, which may optionally comprise one or more heteroatoms,
where the radicals R1, R2, R3, R4 and R5 may optionally be substituted one or more times by alkyl, phenyl, halogen, alkyl carboxylate, O-protected hydroxy and hydroxyalkyl groups, and
R6 is H or an N-protective group, is converted
a) by ozonolysis in a solvent and
b) subsequent decomposition of the peroxide-containing solution using an oxidizing agent or reductive work-up into the corresponding amino compound of the formula 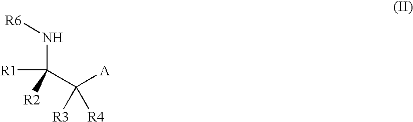
   in which R1, R2, R3, R4 and R6 are as defined above, and A is a radical of the formula —COOH, —C(OH)R5 or —C(O)R5, where R5 is as defined above, depending on the work-up.

In the method of the invention, beta-amino acids, aldehydes or ketones or gamma-amino alcohols are prepared starting from allyl compounds of the formula (I).

In the formula (I), R1 is an alkyl radical, a cyclo-alkyl radical, an aryl radical, a heterocyclic radical or a fused or bridged ring system,
R2, R3, R4 and R5 are independently of one another H or an alkyl radical, a cycloalkyl radical, an aryl radical, a heterocyclic radical or a fused or bridged ring system.

The radicals R1, R2, R3 and R4 may optionally also form ring systems among one another, which may optionally comprise one or more heteroatoms. Thus, for example, a ring system may be formed by R1 with R2 or with R3 or with R4, or R2 with R3 or R4 or R3 with R4. A further possibility is for these ring systems to comprise one or more heteroatoms from the group of O, N or S.

Alkyl radical, for example C₁-C₂₀-alkyl radicals, mean in this connection linear or branched alkyl radicals such as, for instance, methyl, ethyl, n-propyl, i-propyl, n-butyl, sec-butyl, tert-butyl, hexyl, etc. C₂-C₆-alkyl radicals are preferred in this connection. Cycloalkyl radicals are cyclic alkyl radicals, for example cycloalkyl radicals having 3-12 C atoms, such as, for instance, cyclopropyl, cyclohexyl, cyclooctyl, etc. C₃-C₆-Cycloalkyl radicals are preferred.

Suitable aryl radicals are aromatic rings and ring systems having, for example, 5 to 20 C atoms, such as, for instance, phenyl, naphthyl, indenyl, fluorenyl, etc. Preferred aryl radicals are C₆-C₁₀-aryl radicals.

Heterocyclic radicals mean cyclic radicals having, for example, 4 to 20 C atoms, which may comprise at least one heteroatom from the group of O, S or N and may be aromatic or saturated or unsaturated aliphatic rings, such as, for instance, pyrryl, furanyl, thienyl, pyridyl, pyrimidinyl, thiazolyl, indolyl, purinyl, tetrahydrofuranyl, dihydrofuranyl, thiolanyl, piperidinyl, dihydropyranyl, morpholinyl, etc.

C₄-C₁₀-Heterocycles having one to two heteroatoms from the group of O, S or N are preferred in this connection.

Fused ring systems, for example having 6 to 20 C atoms, consist of two or more fused rings, where the rings may be aliphatic or aromatic and may optionally comprise one or more heteroatoms from the group of N, S or O. Examples are, for instance, indane, tetralin, quinoline, chroman, decalin, etc.

Bridged ring systems are, for example, bicyclo[2.2.1]heptane, bicyclo[2.2.2]octane, etc.

The radicals may optionally be substituted one or more times. Suitable substituents in this connection are alkyl, for example C₁-C₄-alkyl, phenyl, halogen, alkyl carboxylate, for example C₁-C₆-carboxylic esters having 1 to 4 C atoms in the ester moiety, O-protected hydroxy and-hydroxyalkyl groups.

Preferred substituents are C₁-C₂-alkyl, phenyl, fluorine, chlorine, C₁-C₂-alkyl C₁-C₃-carboxylate, and hydroxy and hydroxy-C₁-C₄-alkyl groups protected by an acetyl group.

R1 particularly preferably is a phenyl or naphthyl radical or C₂-C₆-alkyl radical, each of which is optionally substituted once or twice by fluorine, chlorine, C₁-C₂-alkyl C₁-C₃-carboxylate or hydroxy or hydroxy-C₁-C₄-alkyl group protected by an acetyl group, or a fused ring system having 6-10 C atoms. R2, R3 and R4 are R5 is particularly preferably H or a C₁-C₆-alkyl radical.

R6 may in formula (I) be H or an N-protective group. Suitable N-protective groups are all conventional N-protective groups such as, for instance, acetyl, formyl, chloroacetyl, trichloroacetyl, phenylacetyl, picolinoyl, benzoyl, carbamates such as, for example, methyl, ethyl, 9-fluoroenylmethyl, 2,2,2-trichloro-ethyl, or other protective groups for amines as described for example in Theodora W. Greene, Peter G. M. Wuts Protective Groups in Organic Synthesis; Third Edition, Wiley Interscience.

The allyl compounds of the formula (I) are converted according to the invention in two steps, by ozonolysis and subsequent oxidative or reductive work-up, into the corresponding amino compounds of the formula (II).

The radicals R1, R2, R3, R4 and R6 in formula (II) are as defined above. The radical A is either a carboxyl group, so that the compound of the formula (II) is a beta-amino acid, or a —C(OH)R5 group in which R5 is as defined above, so that the compound of the formula (II) is a gamma-amino alcohol, or a —C(O)R5 group in which R5 is as defined above, so that the compound of the formula (II) is a beta-amino aldehyde or ketone.

The ozonolysis in the first step takes place in a solvent. Examples of suitable solvents are C₁-C₆-carboxylic acids, water/sulfuric acid mixture, C₁-C₄-alcohol, ethyl acetate or butyl acetate or mixtures thereof.

The reaction temperature is adjusted depending on the chosen solvent and is preferably −40 to +30° C.

If beta-amino acids of the formula (II) with A equal to —COOH are the desired final products, the ozonolysis of the compound of the formula (I) in which R5 is H preferably takes place in a solvent from the C₁-C₆-carboxylic acid group or in a water/sulfuric acid mixture.

For this purpose, the compound of the formula (I) is first taken up in a C₁-C₆-carboxylic acid or in a water/sulfuric acid mixture in the ratio from 10:1 to 50:1, and the reaction solution obtained in this way is equilibrated at a temperature of from 0 to 30° C.

Carboxylic acids preferably employed in this connection are acetic acid or propionic acid.

The reaction with ozone then takes place, supplying ozone in an amount of from 1 to 2 equivalents in the form of an ozone/oxygen stream.

If the gamma-amino alcohols of the formula (II) with A equal to C(OH)R5 or beta-amino aldehydes or ketones of the formula (II) with A equal to C(O)R5 are the desired final products, the ozonolysis of the compound of the formula (I) preferably takes place and is taken up in a C₁-C₆-alcohol or in butyl acetate or ethyl acetate or mixtures thereof, and the reaction solution obtained in this way is equilibrated at a temperature of from -40 to 0C, preferably at −30 to −10° C.

The alcohol preferably employed in this case is methanol or butanol.

The reaction with ozone then takes place, supplying ozone in an amount of from 1 to 2 equivalents in the form of an ozone/oxygen stream.

In the second step, the reaction solution obtained in step one is worked up.

This can take place either by decomposing the peroxide-containing solution with an oxidizing agent or by reductive work-up.

If the beta-amino acid is the desired final product, completion of the ozonolysis is followed by heating the peroxide-containing reaction mixture preferably to 25° C. to the boiling point of the solvent, preferably to 50 to 70° C., and adding from 1 to 10 equivalents, preferably 4 to 8 equivalents, of an oxidizing agent. Suitable oxidizing agents are conventional oxidizing agents such as, for example, H₂O₂, tert-butyl hydroperoxide or oxygen. H₂O₂ in the form of a 30 to 70% strength solution is preferably employed.

After the peroxide decomposition is complete, the solvent/water mixture is distilled off and the desired beta-amino acid is purified where appropriate by recrystallization or column chromatography. In the case of a sulfuric acid/water mixture, completion of the reaction is followed by adjustment of the pH with alkali (e.g. NaOH) so that the isoelectric point of the particular amino acid is reached. The amino acid then precipitates and is filtered off, washed with water and dried.

The desired beta-amino acids are in this case obtained in yields of up to 99% of theory. The enantiomeric excess of the beta-amino acids obtained in this way corresponds to that of the employed compound of the formula (I).

If the gamma-amino alcohols are the desired final products, completion of the ozonolysis is followed by a reductive work-up of the resulting reaction solution in the presence of a reducing agent.

The reductive work-up is in this case preferably carried out with a reducing agent from the group of NaBH₄ or a complex hydride. Examples of reducing agents which can thus be employed are NaBH₄, (R)-Alpine borane®, L-Selectride® or other complex hydrides with or without chiral ligands.

This entails the reaction solution being added to an alcoholic solution which comprises the reducing agent. The alcohol preferably employed for the alcoholic sodium borohydride solution is the alcohol also used as preferred solvent for the ozonolysis.

The amount of reducing agent in this case is from 0.5 to 4 mol per mol of allyl compound of the formula (I). Preferably from 0.5 to 2 mol per mol of allyl compound of the formula (I) are employed.

The reaction solution is then warmed to 10 to 40° C., preferably to 20 to 30° C., and 1-2 equivalents of water, based on the reducing agent, are added in order to decompose excess reducing agent.

The solvent is then distilled off, and the residue is extracted one to five times by usual extractants such as, for instance, dichloromethane, ethyl acetate, butyl acetate, MTBE. The combined organic phases are dried, filtered and finally freed of extractant. The beta-amino alcohols can where appropriate also be purified by recrystallization or column chromatography.

The desired gamma-amino alcohols are in this case obtained in yields of up to 93% of theory. The enantiomeric excess of the gamma-amino alcohols obtained in this way corresponds to that of the employed compound of the formula (I).

If the beta-amino aldehydes or ketones of the formula (IV) are the desired final products, completion of the ozonolysis is likewise followed by a reductive work-up of the resulting reaction solution.

The reductive work-up can in this case take place for example with hydrogen in the presence of a hydrogenation catalyst. The catalytic hydrogenation can moreover be carried out in analogy to the prior art, for example in analogy to EP 147593; EP 99981 or EP 1366008.

The hydrogenation in this case takes place in an organic diluent which is inert under the hydrogenation reaction conditions. Organic diluents mean in this connection besides the solvent used in the ozonolysis, conventional diluents used in hydrogenation, such as, for example, aliphatic or aromatic, optionally chlorinated hydrocarbons, such as pentane, hexane, cyclohexane, toluene, xylenes, methylene chloride, dichloroethane, chlorobenzenes, carboxylic esters such as methyl, ethyl, or butyl acetate, ethers and ketones, as long as they are unable to form peroxides which are a safety concern, and alcohols such as methanol, ethanol, isopropanol.

Suitable catalysts are the noble metal catalysts normally used for hydrogenations, which can be employed in the form of powder catalysts with support materials or without support material. Palladium or platinum catalysts are preferably used, in particular platinum catalysts without support material. In the case of powder catalysts, a suitable support material are for example, carbon, aluminum, silica gel or kieselguhr. The amount of hydrogen which can be used in the hydrogenation extends from one mole equivalent up to a multiple molar excess. The use of excess hydrogen has no intrinsic advantages and is only expedient in order to ensure an adequate supply of hydrogen to the hydrogenation mixture.

The hydrogenation advantageously takes place in the method according to the invention under virtually atmospheric pressure. Virtually atmospheric pressure is intended to mean here pressures of from 1 to about 8 bar, as is usual in the art in order to prevent air from entering the hydrogenation reactor. The reductive cleavage proceeds exothermically and is carried out at 15 to 40° C., preferably at temperatures in the range from 20 to 40° C.

The reaction mixture after completion of the hydrogenation is worked up by removing the catalyst by one of the known methods, for example by filtration, decantation or centrifugation and the solvent is preferably recovered by distillation.

The reductive work-up can, however, also take place by reduction using triphenylphosphine, tributylphosphine, thiourea, organic sulfides such as, for example, dimethyl sulfide or bisethanol sulfide, or using zinc in acetic acid.

The desired beta-amino aldehydes or ketones are in this case obtained in yields of up to 90% of theory. The enantiomeric excess of the beta-or gamma-amino alcohols obtained in this way corresponds to that of the employed compound of the formula (I).

If the resulting aldehyde or ketone is insufficiently stable, it can be converted into the acetal or ketal or into a bisulfite adduct. Protection of the aldehyde or ketone can also be carried out in solution which is obtained after the ozonolysis and hydrogenation. The protective group can be introduced in accordance with the state of the literature as described for example in: Theodora W. Greene and Peter G. M. Wuts; Protective Groups in Organic Synthesis, Third Edition, Wiley Interscience, 1999.

### EXAMPLE 1-6

Procedure for Preparing Chiral Amino Alcohols

0.04 mol of unprotected or protected allylamine were taken up in 200 ml of methanol. The solution was put into a jacketed vessel and cooled to −20° C. After a constant ozone/oxygen stream of 20 g/Nm³ was adjusted, the ozonolysis was started. After the ozonolysis was complete, the reaction solution was added dropwise to an ice-cooled methanolic sodium borohydride solution (0.09 mol, 100 ml) over the course of 10 minutes. The reaction solution is then warmed to room temperature and then 10 ml of water are added in order to decompose excess sodium borohydride. The solvent was then distilled off, and the residue was extracted with dichloromethane several times. The combined organic extracts were dried over sodium sulfate, filtered and then the solvent was distilled off.

The resulting product was purified where appropriate by recrystallization or column chromatography.

### EXAMPLE 1

Starting compound: (R)-4-Amino-4-phenyl-1-butene

Product: (R)-3-Amino-3-phenyl-1-propanol was obtained in a yield of 93% and an enantiomeric excess of 99%

White crystals; mp 73-74° C.;

¹H-NMR (CDCl₃) 1.86 (m, 2H, —CH₂CH₂OH), 3.76 (t, 2H, —CH₂CH₂OH), 4.10 (t, 1H, —CH₂CH₂OH), 7.21-7.35 (m, 5H, Ar—H)

### EXAMPLE 2

Starting compound: (R)-4-Amino-4-(4-pyridyl)-1-butene

Product: (R)-3-Amino-3-(4-pyridyl)-1-propanol

Yield (after column chromatography): 69%

Enantiomeric excess 40%

Yellow oil; [α]_{D}=33.24 (c=1.02 g/ml, chloroform);

¹H-NMR (CDCl₃) 1.95 (s, 3H, Ac—CH₃), 2.07 (s, 3H, Ac—CH₃), 2.18 (m, 2H, —CH₂CH₂OAc), 4.00-4.20 (m, 2H, —CH₂CH₂OAc), 5.13 (m, 1H, —CH(NHAC)—), 7.26-8.50 (m, 4H, Pyr-H), 8.61 (br s, 1H, -NHAc) ppm;

¹³C-NMR (CDCl₃) 20.8 (Ac—CH₃), 22.9 (Ac—CH₃), 34.5 (—CH₂CH₂OAc), 48.5 (—CH(NHAc)—), 60.9 (—CH₂CH₂OAc), 123.9 (Pyr-C), 134.5 (Pyr-C), 138.0 (Pyr-C), 148.4 (Pyr-C), 148.5 (Pyr-C), 170.2 (Ac—CO), 170.9 (Ac—CO) ppm.

### EXAMPLE 3

Starting compound: (R) -4-Amino-4-(4-fluorophenyl)-1-butene

Product: (R)-3-Amino-3-(4-fluorophenyl)-1-propanol

Yield: 83%

Enantiomeric excess 87%:

White crystals; mp 141-142° C.; [α]_{D} =22.61 (c=1.99 g/ml, chloroform);

¹H-NMR (CDCl₃) 1.84 (m, 2H, —CH₂CH₂OH), 3.17 (br s, 3H, —OH, —NH₂), 3.70 (m, 2H, CH₂CH₂OH), 4.10 (t, 1H, —CH(NH₂)—), 6.91-7.29 (m, 4H, Ar—H) ppm;

¹³C-NMR (CDCl₃) 40.2 (—CH₂CH₂OH), 54.8 (—CHNH₂), 60.7 (—CH₂CH₂OH), 112.8-113.2 (Ar—C Pos. 4), 113.8-114.2 (Ar—C Pos. 6), 121.7 (Ar—C Pos. 3), 129.8 (Ar—C Pos. 2), 148.6 (Ar—C Pos. 1), 161.4-164.7 (Ar—C Pos. 5) ppm.

### EXAMPLE 4

Starting compound: (R) -N-Acetyl-4-amino-4-phenyl-2-methyl-1-butene

Product: (R,±)-N-Acetyl-4-amino-4-phenyl-2-butanol

Yield: 84%

White solid: mp 87-88° C.; [α]_{D} =100.54 (c=1.85 g/ml, chloroform);

¹H-NMR (CDCl₃) 1.16 (d, 3H, —CH₂CH(CH₃)OH), 1.78 (m, 2H, —CH₂CH(CH₃)OH), 1.95 (s, 3H, Ac—CH₃), 3.76 (m, 1H, —CH₂CH(CH₃)OH), 4.01 (br s, 1H, —OH), 4.95-5.18 (ddd, 1H, —CH(NHAc)—), 7.07 (m, 1H, —NHAc), 7.20-7.32 (m, 5H, Ar—H) ppm;

¹³C-NMR (CDCl₃) □ 23.0 (—CH(CH₃)OH), 23.1 (Ac—CH₃), 45.2 (—CH₂CH(CH₃)OH), 51.0 (—CH(NHAc)—), 63.9 (—CH₂CH(CH₃)OH), 126.6 (Ar—C), 127.4 (Ar—C), 128.6 (Ar—C), 141.5 (Ar—C), 171.0 (Ac—CO) ppm. EXAMPLE 5

Starting compound: (R)-N-Acetyl-4-amino-4-phenyl-2-methyl-1-butene

Product: (R,R)-N-Acetyl-4-amino-4-phenyl-2-butanol Reduction of the peroxide solution was carried out with (R)-Alpine borane® and with L-Selectride® in analogy to the reduction with sodium borohydride.

Yield after recrystallization (from acetonitrile) 76%.

L-Selectride®: diastereomer ratio 1:3

(R)-Alpine borane®: diastereomer ratio 1:2.

Colorless oil; [α]_{D}=53.88 (c=2.06 g/ml, chloroform);

¹H-NMR (CDCl₃)1.07 (m, 3H, —CH(CH₃)OH), 1.43-1.71 (m, 2H, —CH₂CH(CH₃)OH), 1.89 (s, 3H, Ac—CH₃), 3.51 (m, 1H, —CH(CH₃)OH), 4.32 and 4.49 (2 s, 1H, —OH), 4.94 (m, —CH(NHAc)—), 7.20-7.37 (m, 5H, Ar—H), 8.21-8.30 (2 d, 1H, -NHAc) ppm.

### EXAMPLE 6

Starting compound: (R)-N-Acetyl-4-amino-5-methyl-1-hexene

Product: (R) -N-Acetyl-3-amino-4-methyl-1-pentanol

Yield: 93%

Enantiomeric excess: 89%

White crystals: mp 67° C.; [α]_{D}=11.29 (c=18.6 g/ml, chloroform);

¹H-NMR (CDCl₃) 0.94 (dd, 6H, —CH(CH₃)₂), 1.33 (m, 1H, —CH(CH₃)₂), 1.71-1.87 (m, 2H, —CH₂CH₂OH), 2.03 (s, 3H, Ac—CH₃), 3.57 (m, 2H, —CH₂CH₂OH), 3.83 (m, 1H, —CH(NHAc)—), 3.96 (br s, 1H, —CH₂CH₂OH), 6.04 (d, 1H, CH—NHAc)—) ppm;

¹³C-NMR (CDCl₃) 18.9 (—CH(CH₃)₂), 19.7 (—CH(CH₃)₂), 23.4 (Ac—CH₃), 32.3 (—CH(CH₃)₂), 35.5 (—CH₂CH₂OH), 51.9 (—CH(NHAc)—), 59.2 (—CH₂CH₂OH), 172.0 (Ac—CO) ppm.

Procedure for Preparing Amino Aldehydes/Ketones

0.04 mol of unprotected or protected allylamine were taken up in 200 ml of methanol. The solution was put into a jacketed vessel and cooled to −20° C. After a constant ozone/oxygen stream of 20 g/Nm³ was adjusted, the ozonolysis was started. After the ozonolysis was complete, the reaction solution was hydrogenated with hydrogen and a hydrogenation catalyst, e.g. Pd/C (5%) under atmospheric pressure at 25° C.

The hydrogenation catalyst was then removed by filtration, and the solvent was distilled off.

The resulting product was purified where appropriate by recrystallization or column chromatography.

### EXAMPLE 7

Starting compound: (R)-N-Acetyl-4-amino-4-phenyl-2-methyl-1-butene

Product: (R)-N-Acetyl -4 -amino-4-phenylbutane-2-one

Yield: 76%

Enantiomeric excess: 97%:

White crystals; mp 77-78° C.; [α]_{D}=50.94 (c=2.12 g/ml, chloroform);

¹H-NMR (CDCl₃) 1.95 (d, 3H, —CO—CH₃), 2.07 (s, 3H, Ac—CH₃), 2.77-3.07 (ddd, 2H, —CH₂COCH₃), 5.37 (dd, 1H, —CH(NHAc)—), 7.04 (d, 1H, —NHAc), 7.20-7.32 (m, 5H, Ar—H) ppm;

¹³C NMR (CDCl₃) 23.2 (Ac—CH₃), 30.5 (—CH₂COCH₃), 48.6 (—CH₂COCH₃) , 49.6 (—CH(NHAc)—), 126.4 (Ar—C), 127.7 (Ar—C), 128.9 (Ar—C), 141.1 (Ar—C), 169.6 (Ac—CO), 207.3 (—COCH₃) ppm.

### EXAMPLES 8-11

Procedure for Preparing Chiral Beta-amino Acids

0.02 mol of acetyl-protected allylamine was taken up in 200 ml of acetic acid (technical quality) . The reaction solution was equilibrated at 18° C. It was ozonolyzed with an ozone/oxygen stream with an ozone concentration of 20 g/Nm³/h. After the ozonolysis was complete, the reaction mixture was heated to 60° C., and 6 equivalents of hydrogen peroxide (50%, technical quality) were added. The reaction was complete after 2-3 hours. The acidic acid/water mixture was distilled off. The product was purified if necessary by recrystallization from acetonitrile or by column chromatography.

### EXAMPLE 8

Starting compound: (R)-4-N-Acetylamino-4-phenyl-1-butene

Product: (R)-N-Acetyl-β-phenyl-β-alanine

Yield: 97%

Enantiomeric excess: 99%

White crystals; mp 198° C.; [α]_{D}=84.30 (c=1.5 g/ml, ethanol);

¹H-NMR (DMSO-d₆) 1.83 (s, 3H, Ac—CH₃), 2.68 (m, 2H), 5.21 (m, 1H), 7.21-7.33 (m, 5H), 8.38 (d, 1H), 12.26 (br s, 1H) ppm;

¹³C-NMR (DMSO-d₆) 23.0 (Ac—CH₃), 41.3 (—CH₂—COOH), 49.8 (—CH(NHAc)—), 126.8 (Ar—C), 127.3 (Ar—C), 128.6 (Ar—C), 143.0 (Ar—C), 168.6 (—COOH), 172.1 (Ac—CO) ppm.

### EXAMPLE 9

Starting compound: (R)-4-N-Acetylamino-4-(4-pyridyl)-1-butene Product: (R)-N-Acetyl-3-(4-pyridyl) -3-aminopropionic acid

Yield: 96%

Enantiomeric excess: 40%

Pale yellow crystals; mp 82-83° C.; [α]_{D}=8.24 (c=2.55 g/ml, water);

¹H-NMR (CDCl₃) 1.89 (s, 3H, Ac—CH₃), 2.36 (t, 2H, —CH₂COOH), 5.00-5.15 (m, 1H, —CH(NHAc)—), 7.30-8.55 (m, 4H, Pyr-H), 8.93 (m, 1H, -NHAc) ppm;

¹³C-NMR (CDCl₃) 24.3 (Ac—CH₃), 26.9 (—CH₂COOH), 50.3 (—CH(NHAc)—), 124.6 (Pyr-C), 135.5 (Pyr-C), 148.8 (Pyr-C), 149.8 (Pyr-C), 169.6 (Ac—CO), 176.4 (—COOH) ppm.

### EXAMPLE 10

Starting compound: (R)-4-N-Acetylamino-4-(4-fluorophenyl) -1-butene

Product: (R)-N-Acetyl-3-(4-fluorophenyl)-3-amino-propionic acid

Yield: 99%

Enantiomeric excess: 89%:

White crystals; mp 33° C.; [α]_{D}=69.60 (c=2.27 g/ml, methanol);

¹H-NMR (CDCl₃) 1.85 (s, 3H, Ac—CH₃) , 2.67 (d, 2H, —CH₂COOH), 5.22 (m, 1H, —CH(NHAc)—), 7.01-7.39 (m, 5H, Ar—H), 8.40 (d, 1H, —CH(NHAc)—) ppm;

13C-NMR (CDCl₃) 23.4 (Ac—CH₃), 41.6 (—CH₂COOH), 50.0 (—CH(NHAc)—), 114.0 (Ar—C), 114.4 (Ar—C), 123.4 (Ar—C), 130.9 (Ar—C), 146.5 (Ar—C), 161.4-164.6 (Ar—C), 169.3 (Ac—CO), 172.4 (—COOH) ppm.

### EXAMPLE 11

Starting compound: (R)-N-Acetyl-4-amino-5-methyl-1-hexene

Product: (R)-N-Acetyl-3-amino-4-methylpentanoic acid

Yield after column chromatography: 58%

Enantiomeric excess: 98%

Yellow crystals; mp 84-85° C.; [α]_{D}=−29.03 (c=2.17 g/ml, chloroform);

¹H-NMR (CDCl₃) 0.93 (s, 6H, —CH(CH₃)₂), 2.00 (m, 3H, Ac—CH₃), 2.56 (s, 2H, —CH₂—COOH), 4.05 (s, 1H, —CH(NHAc)—), 6.74 (br s, 1H, -NHAc) ppm;

¹³C-NMR (CDCl₃) 18.9 (—CH(CH₃)₂), 19.2 (—CH(CH₃)₂), 23.0 (Ac—CH₃), 40.2 (—CH₂—COOH), 51.9 (—CH(NHAc)—), 171.3 (Ac—CO), 175.7 (CH₂—COOH) ppm.

1. An improved method for preparing chiral or enantiomer-enriched beta-amino acids, aldehydes, ketones or gamma-amino alcohols, characterized in that an allylamine of the formula 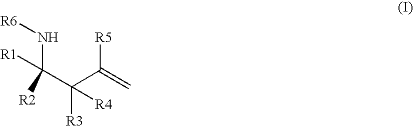
in which R1 is an alkyl radical, a cycloalkyl radical, an aryl radical, a heterocyclic radical or a fused or bridged ring system,
R2, R3, R4 and R5 may independently of one another be H or an alkyl radical, a cycloalkyl radical, an aryl radical, a heterocyclic radical or a fused or bridged ring system,
or the radicals R1, R2, R3 and R4 may form ring systems among themselves, which may optionally comprise one or more heteroatoms,
where the radicals R1, R2, R3, R4 and R5 may optionally be substituted one or more times by alkyl, phenyl, halogen, alkyl carboxylate, O-protected hydroxy and hydroxyalkyl groups, and R6 is H or an N-protective group, is converted
a) by ozonolysis in a solvent and
b) subsequent decomposition of the peroxide-containing solution using an oxidizing agent or reductive work-up
into the corresponding amino compound of the formula 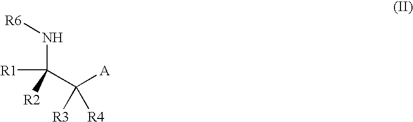
in which R1, R2, R3, R4 and R6 are as defined above,
and A is a radical of the formula —COOH, —C(OH)R5 or —C(O)R5, where R5 is as defined above, depending on the work-up.

2. The method as claimed in claim 1, characterized in that R1 in the formula (I) is a C₁-C₂₀-alkyl radical, a C₃-C₁₂-cycloalkyl radical, a C₅-C₂₀-aryl radical, a C₄-C₂₀-heterocyclic radical or a fused or bridged ring system having 6 to 20 C atoms,
R2, R3, P.4 and R5 may independently of one another be H or a C₁-C₂₀-alkyl radical, a C₃-C₁₂-cycloalkyl radical, a C₅-C₂₀-aryl radical, a C₄-C₂₀-heterocyclic radical or a fused or bridged ring system having 6 to 20 C atoms,
or the radicals R1, R2, R3 and R4 may form C₃-C₁₀ ring systems among one another, which may optionally comprise one or more heteroatoms, where the radicals R1, R2, R3, R4 and R5 may optionally be substituted one or more times by C₁-C₄-alkyl, phenyl, halogen, C₁-C₄-alkyl C₁-C₁₆-carboxylate, 0-protected hydroxy and hydroxyalkyl groups, and R6 is H or an N-protective group.

3. The method as claimed in claim 1, characterized in that step a) is carried out in a solvent from the group of C₁-C₆-carboxylic acid, water/sulfuric acid mixture, C₁-C₄-alcohol, ethyl acetate or butyl acetate or mixtures thereof.

4. The method as claimed in claim 1, characterized in that the reaction temperature in step a) is from −40° C. to +30° C., depending on the chosen solvent.

5. The method as claimed in claim 1, characterized in that the ozonolysis for the allyl of the formula (I) in which R5 is H. is carried out in a C₁-C₆-carboxylic acid or in a water/sulfuric acid mixture in the ratio from 10:1 to 50:1 as solvent at a temperature of from 0 to 30° C.

6. The method as claimed in claim 5, characterized in that acetic acid or propionic acid is employed as solvent for the ozonolysis.

7. The method as claimed in claim 1, characterized in that if gamma-amino alcohols of the formula (II) with A equal to C(OH)R5 or beta-amino aldehydes or ketones of the formula (II) with A equal to C(O)R5 are the desired final products, the ozonolysis takes place in a C₁-C₆-alcohol or in butyl acetate or ethyl acetate or in mixtures thereof as solvent at a temperature from −40° C. to 0° C.

8. The method as claimed in claim 7, characterized in that methanol or butanol is employed as solvent for the ozonolysis.

9. The method as claimed in claim 1, characterized in that an oxidizing agent from the group of H₂O₂, tert-butyl hydroperoxide and oxygen is employed in an amount of from 1 to 10 equivalents for the decomposition of the peroxide-containing solution resulting from step a) by means of an oxidizing agent, and the solution is heated to 25° C. to the boiling point of the solvent.

10. The method as claimed in claim 1, characterized in that if beta-amino acids of the formula (II) with A equal to —COOH are the desired final product, the decomposition of the peroxide-containing solution resulting from step a) takes place by means of an oxidizing agent.

11. The method as claimed in claim 1, characterized in that if beta-amino acids of the formula (II) with A equal to —COOH are the desired final product, the ozonolysis is carried out in a C₁-C₆-carboxylic acid or in a water/sulfuric acid mixture in the ratio from 10:1 to 50:1 as solvent at a temperature of from 0 to 30° C., and the work-up of the ozonolysis solution includes an oxidizing agent from the grout of H₂O₂, tert-butyl hydroperoxide and oxygen which is employed in an amount of from 1 to 10 equivalents for the decomposition of the peroxide-containing solution resulting from step a), and the solution is heated to 25° C. to the boiling point of the solvent.

12. The method as claimed in claim 10, characterized in that after the peroxide decomposition is complete, the solvent/water mixture is distilled off and the beta-amino acid is purified where appropriate by recrystallization or column chromatography.

13. The method as claimed in claim 1, characterized in that in the case where amino alcohols of the formula (II) with A equal to C(OH)R5 are the desired final compounds, a reducing agent from the group of NaBH₄ or of the complex hydrides is employed for the reductive work-up in step b).

14. The method as claimed in claim 13, characterized in that NaBH₄, (R)-Alpine borane®, L-Selectride® or other complex hydrides with or without chiral ligands are employed as reducing agent.

15. The method as claimed in claim 13, characterized in that from 0.5 to 4 mol of reducing agent are employed per mol of allyl compound of the formula (I).

16. The method as claimed in claim 13, characterized in that after the reductive work-up is complete, the reaction solution is warmed to 10 to 40° C., and 1 to 2 equivalents of water based on the reducing agent, are added in order to decompose excess reducing agent.

17. The method as claimed in claim 13, characterized in that the gamma-amino alcohol is isolated by extraction, with the beta-amino alcohol also being purified where appropriate by recrystallization or column chromatography.

18. The method as claimed in claim 1, characterized in that if beta-amino aldehydes or ketones of the formula (II) with A equal to C(O)R5 are the desired final products, the reductive work-up in step b) takes place with hydrogen in the presence of a hydrogenation catalyst or by reduction with triphenylphosphine, or tributylphosphine, thiourea, organic sulfides or by zinc in acetic acid.

19. The use of the beta-amino acides, aldehydes, ketones or gamma-amino alcohols prepared as claimed in claim 1 as intermediates for pharmaceutical products.
